(19) United States
(12) Patent Application Publication
(10) Pub. No.: US 2019/0262151 A1
(43) Pub. Date: Aug. 29, 2019

(54) WOVEN STENT DEVICE WITH CAPPED ENDS AND MANUFACTURING METHOD

(71) Applicant: Cook Medical Technologies LLC, Bloomington, IN (US)

(72) Inventor: Gerard Treacy, Limerick (IE)

(73) Assignee: Cook Medical Technologies LLC, Bloomington, IN (US)

(21) Appl. No.: 16/285,564

(22) Filed: Feb. 26, 2019

Related U.S. Application Data
(60) Provisional application No. 62/636,361, filed on Feb. 28, 2018.

(51) Int. Cl. A61F 2/90

(52) U.S. Cl. CPC A61F 2/90; A61F 2/06

(57) ABSTRACT

A stent and method for treating an end of a pre-woven stent is provided. The method includes providing a pre-woven stent including a plurality of sharp wire ends. The wire ends are arranged in pairs. A separate cap is placed over the ends of the paired wires. The wire ends are secured to the cap, such that the sharp ends are covered by the cap. The cap may have a pair of leg portions that combine to form a curved bend portion. The cap may be flexible. The wire ends may be received in open ends of the cap. The cap may include a pair of legs portions connected by a flexible bridge portion. The caps may be disposed at different axial locations. The caps may be disposed at a single end of the stent, with the opposite end being without sharp ends after being pre-woven. Alternatively, the caps may be applied to both ends of the pre-woven stent having sharp ends at both ends after being pre-woven. The pre-woven stent is preferably a machine woven stent.

### CROSS-REFERENCE TO RELATED APPLICATIONS

This application claims priority to U.S. provisional patent application No. 62/636,361 filed on Feb. 28, 2018, the entirety of which is hereby incorporated by reference.

### TECHNICAL FIELD

The present disclosure relates generally to medical devices, and more particularly to stents having soft crowns.

### BACKGROUND

Stents are medical devices commonly used to maintain patency of diseased body vessels, such as those of the vascular and gastrointestinal systems. Stents are often delivered via a minimally invasive procedure and thereafter expanded to contact and support the inner wall of the targeted vessel. In general, most stents include a tubular shaped support structure having a plurality of interstices configured to facilitate compression and expansion of the stent.

Many stents define the tubular shaped structure by weaving a plurality of strands together in a traditional weave pattern, where the strands overlap each other in an alternating fashion, with two major weaving directions. The weaving directions are ultimately in a helical shape, with a first set of strands extending around the stent in the first helical direction, and a second set of strands extending around the stent in a second helical direction that is transverse to the first helical direction.

Many stents define a proximal and distal end of the woven tubular structure, where the ends of the strands terminate to define the proximal and distal ends. The strands are typically in the form of individual wire, so the terminal ends are generally sharp at the proximal and distal ends.

In many instances, the stents include proximal and distal flanges or flared ends to prevent stent migration subsequent to implantation. Flanges or flares are typically set to a larger expanded diameter relative to the stent central portion and may exert a higher radial force per unit area against the vessel wall, thereby securing the stent in position. One problem with these features, however, is that the flanges or flares can damage the vessel wall if they are excessively rigid, especially in light of the sharp ends of the strands. The resulting tissue perforations may be painful and can lead to more serious complications including infection, hemorrhage, and possibly death.

Accordingly, there have been attempts to “treat” the sharp ends of woven stents to reduce the effects of the sharpness of the terminal ends. One method of treating the sharp ends of the woven stent includes welding or soldering a curved member to the sharp ends to join a pair of sharps ends to create a weld joint. However, fatigue stresses become concentrated at the weld joint, which can lead to breaking and exposing of a sharp end, which can lead to injury to the patient.

Another method of addressing the sharp ends of a woven stent can include weaving the stent by hand, such that sharp ends of the wires are not created. For example, a wire may be woven toward an end of the stent, where the wire will be bent and woven back in the opposite direction. When the wire reaches the opposite end, the wire is bent again and the weaving can continue. This type of continuous weaving can result in ends that are bent and without welding or soldering. However, had weaving of stents can be substantially time consuming and costly relative to a more automated weaving system.

### SUMMARY

The present disclosure generally provides a stent with treated ends and a method for treating the ends of a stent.

Other devices, systems, methods, features and advantages will be, or will become, apparent to one with skill in the art upon examination of the following figures and detailed description. It is intended that all such additional devices, systems, methods, features and advantages be included within this description, and be protected by the following claims.

### BRIEF DESCRIPTION OF THE DRAWINGS

The system may be better understood with reference to the following drawings and description. The components in the figures are not necessarily to scale, with emphasis instead being placed upon illustrating the principles of the present disclosure. Moreover, in the figures, like referenced numerals designate corresponding parts throughout the different views.

FIG. 1 shows a machine woven stent having loose wires with untreated ends at one end;

FIG. 2 shows a machine woven stent having loose wires with untreated ends at both ends;

FIG. 3 shows a machine woven stent prior to untangling the loose wires;

FIG. 4 shows a machine woven stent after untangling and pairing the loose wires;

FIG. 5 shows one example of pairing two of loose wires;

FIG. 6 shows another example of pairing two of the loose wires;

FIG. 7 shows a cap for being placed over the ends of a pair of loose wires to treat the sharp ends of the wires, the cap being in the form of a thin walled cannula having a U-shape;

FIG. 8 shows the cap fixed in place over the pair of wires;

FIG. 9 shows another example of the cap;

FIG. 10 shows a membrane coating applied over the interface between the wires and the cap;

FIG. 11 shows a cap having legs with different lengths;

FIG. 12 shows a flat end of one of the legs of the cap;

FIG. 13 shows a beveled end of one of the legs of the cap;

FIG. 14 shows a cap having a V-shape;

FIG. 15 shows a non-symmetrical cap;

FIG. 16 shows a bridge cap, where a pair of legs are connected via a bridge portion to define the cap;

FIG. 17 shows another example of the bridge cap;

FIG. 18 shows a coil cap, where the cap is defined by a coiled wire;

FIG. 19 shows a coil bridge cap, where the cap is defined by a pair of legs connected by a coil;

FIG. 20 shows a soft cap that is heat shrunk onto the wires;

FIG. 21 shows another example of the soft cap where the wires overlap each other and the soft cap is heat shrunk over the wires;

FIG. 22 shows a molded cap, where the ends of the wires are received in the molded cap;

FIG. 23 shows a pair of wires having curled ends;

FIG. 24 shows the wires with curled ends being attached to each other via the curled ends;

FIG. 25 show the wires with curled ends being attached to each via a linkage having curled ends;

FIG. 26A shows a plurality of caps disposed over a plurality of wire pairs, the caps being disposed at approximately the same axial location;

FIG. 26B shows a plurality of caps, where the caps are disposed at different axial locations;

FIG. 27 shows a pair of overlapping caps that connect a pair of wires that are not circumferentially adjacent at the end of the stent;

FIG. 28A shows a mandrel for use in treating the ends of the stent having a body portion and a head portion;

FIG. 28B shows the loose wires at the end of the stent being paired and capped while on the mandrel;

FIG. 29 shows the mandrel with the body portion removed and replaced with a reduced diameter portion mandrel for creating a flanged stent;

FIG. 30 shows another mandrel having a collapsible body portion and a pair of head portions;

FIG. 31 shows the mandrel with the collapsible body portion removed, and a reduced diameter portion disposed through the head portions and the stent;

FIG. 32 shows the collapsible body portion of the mandrel having sections allowing it to collapse; and

FIG. 33 shows the collapsible body portion and the head portions, with a core pin extending therethrough to maintain in the collapsible body portion in a non-collapsed state.

### DETAILED DESCRIPTION

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

The term “proximal,” as used herein, refers to a direction that is generally towards a physician during a medical procedure.

The term “distal,” as used herein, refers to a direction that is generally towards a target site within a patient's anatomy during a medical procedure.

The term “biocompatible,” as used herein, refers to a material that is substantially non-toxic in the in vivo environment of its intended use, and that is not substantially rejected by the patient's physiological system. A biocompatible structure or material, when introduced into a majority of patients, will not cause an undesirably adverse, long-lived or escalating biological reaction or response. Such a response is distinguished from a mild, transient inflammation which typically accompanies surgery or implantation of foreign objects into a living organism.

### Devices and Systems

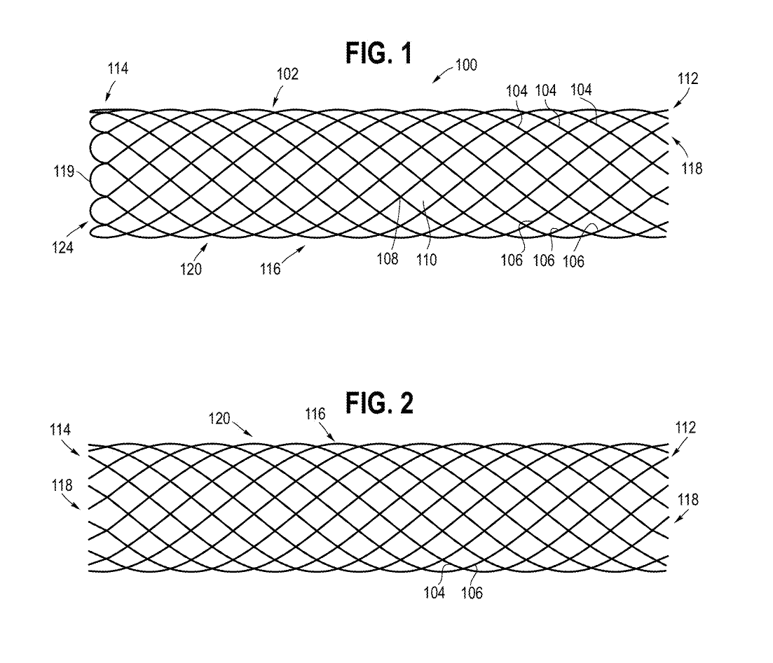

FIG. 1 depicts an embodiment of a woven stent 100. The structure of the stent 100 is formed from a plurality of wires 102 helically wound in an under-over-under configuration. The wires 102 can be defined as a combination of first wires 104 and second wires 106. The first wires 104 extend in a first generally helical direction and the second wires 106 extend in a second generally helical direction.

In one approach, the first and second wires 104, 106 can be separate and distinct wires. In another approach, a pair of the first wires 104 and the second wires 106 can each be portions of a single wire that includes a bend at one end of the stent to define the first wire 104 and second wire 106, with the portion defining the first wire 104 extending in the first helical direction and the portion defining the second wire 106 extending in the second helical direction. For purposes of further discussion, the first and second wires 104, 106 will be described as being separate and distinct wires, unless otherwise noted. But it will be appreciated that the descriptions of these first and second wires 104, 106 can applied to embodiments where pairs of first and second wires 104, 106 are part of one overall bent wire that creates the portions extending in the first and second helical directions.

The first and second helical directions are typically arranged transverse to each other and therefore the first and second wires 104, 106 are likewise arranged transverse to each other. In one approach, the first and second wires 104, 106 are arranged perpendicular to each other at each cross-point or intersection 108 of the wires 104, 106. However, it will be appreciated that the wires 104, 106 can also be arranged at different transverse angles, such that there are both obtuse and acute angles defined by the wires 104, 106 at the intersection 108.

Cross points in the structure (i.e., where the wire crosses over and under itself) further define a plurality of quadrilateral shaped cells 110 defined at their perimeter by the wires 104, 106. The quadrilateral shape of the cell 110 depends on the orientation of the wires 104, 106 at each of the intersections that define the four corners of the cell 110 as well as the spacing between the wires 104, 106. For example, the quadrilateral shape of the cell 110 could be a square, rectangle, rhombus, parallelogram, or the like. It will be appreciated that the woven nature of the stent 100 will allow for various shapes and sizes of the cells 110 as the stent is radially and/or longitudinally compressed and expanded.

The stent 100 includes a first end portion 112 and a second end portion 114, with a tubular body portion 116 extending longitudinally between the first and second end portion 112, 114. The first and second ends 112, 114 can also be referred to as proximal and distal ends.

The body of the stent 100 is defined by the woven structure of the wires, and the overall body can have different shapes, including an enlarged diameter flange portion at one or both ends. It will be appreciated that reference to the stent body can include reference to portions of the stent that are enlarged to define flange portions, and is not limited to the portion that extends between the flanges, unless otherwise noted. Thus, a reference to wires being woven into the body encompasses wires being woven into the flange portion of a stent having flanges, and also to uniform diameter stents, as well as other distances or portions of the stent.

The stent shown in FIG. 1 has a uniform diameter along its length, as is typical for machine woven stents prior to any heat setting or treating. It will be appreciated that other general tubular shapes, including non-uniform diameter or multiple portions having different diameters could also be used, such as a flanged stent where the ends of the stent 100 have a greater diameter than the middle of the stent.

The stent 100 can be manufactured and woven in a variety of ways known to the art. One method of manufacturing involves the use of a mandrel on which the strands of the stent are overlaid and woven together by hand. Hand weaving of entire stents, however, can be costly as well as time-consuming. Another manner of manufacturing the stent involves machine weaving. Machine woven stents can typically be made quickly and at a reduced cost relative to a hand woven stent. Machine woven stents result in the production of a stent having a plurality of sharp wire ends 118 disposed at each of the first and second end portions or, alternatively, at one end. The resulting machine woven stent can be referred to a pre-woven stent 120, as the pre-woven stent 120 has been woven but still includes the undesirable sharp ends 118 at at least one of the first and second end portions 112, 114. The pre-woven stent 120 is shown in FIGS. 1 and 2, and is shown having a uniform diameter. The pre-woven stent 120 of FIG. 1 is shown having the second end portion 114 without sharp ends, as one end of a machine woven stent may be made by bending the wires to define crowns at the end, with the opposite end having sharp ends where the wires terminate. Thus, the pre-woven stent 120 could have just one end having the sharp ends 118, with the wires forming bends 119 that link the first and second wires 104, 106, as described above with reference to a single wire having two portions that define the first and second wires 104, 106. FIG. 2 illustrates the pre-woven stent 120 with sharp ends 118 at both ends of the stent.

Providing the pre-woven stent 120 having sharp wire ends 118 reduces the time for weaving the stent 100 by providing the user with the majority of the stent 100, including the tubular body portion 116. The pre-woven stent 120, however, includes at least one of the first and second end portion 112, 114 in an unfinished condition having the sharp ends 118. However, these sharp ends 118 can be further modified and treated to ultimately produce the finished version of the stent 100 where the sharp ends 118 are eliminated.

For purposes of discussion, the pre-woven stent 120 may be described as having the sharps ends 118 at both the first and second ends 112, 114 or only at a single end. It will be appreciated that references to treating the sharp ends 118 of the wires 104 and 106 can apply to one or both ends of the pre-woven stent, and in the case of only treating one end, it may be assumed that the opposite end does not include sharp ends or has otherwise already been treated to eliminate the sharp ends.

Treating the sharp ends 118 of the pre-woven stent includes converting the sharp ends 118 into a plurality of crowns 124 (an example of the sharp ends 118 being converted in crowns is shown in FIG. 31). Crowns 124 may refer to a curved structure at the end of the stent 100. For example, the bent ends 119 shown in FIG. 1 may also be referred to as crowns 124. The sharp ends 118 may be converted into crowns 124 by being treated as described herein. The crowns 124 may be disposed circumferentially around the first and second ends 112, 114 of the stent 100 after the stent 100 has been treated to create the crowns 124 from the sharp ends 118.

The pre-woven stent 120 can be made either by hand-weaving or machine-weaving. The pre-woven stent 120 includes the plurality of sharp ends 118. The sharp ends 118 are defined by the ends of the wires that are woven together to define the tubular body portion 116. The sharp ends 118 can terminate at approximately the same longitudinal position, or they can terminate at different longitudinal positions.

The pre-woven stent 120 includes the first wires 104 and second wires 106 that extend in transverse helical directions, as described above. The first and second wires 104, 106 are woven together, but typically free to move slightly relative to each other as is typical in a woven structure. Accordingly, each of the wires 104, 106 define the sharp ends 118 at each wire end. The sharp ends 118 are free ends, and capable of being moved and manipulated, as further described below, to treat the sharp ends 118. The sharp ends 118 may be defined as the existing ends of the wires 104 and 106, or they may be created by cutting or trimming the wires 104 and 106 to a desired length or when cutting an elongated pre-woven stent 120 into multiple sections.

The sharp ends 118 of the pre-woven stent 120 are present after the pre-woven stent 120 has been woven. For purposes of discussion, further discussion of the pre-woven stent 120 will refer to a machine woven version.

The pre-woven stent 120 can be machine woven onto a machine mandrel. The machine mandrel typically has a generally cylindrical shape, resulting in a generally cylindrical pre-woven stent 120. This arrangement can also be referred to as parallel. At the conclusion of the machine weaving process (which will not be described in detail), the machine mandrel 125, having the pre-woven stent 120 woven thereon, can be removed from the weaving/braiding machine and the stent 120 may remain on the mandrel for further processing.

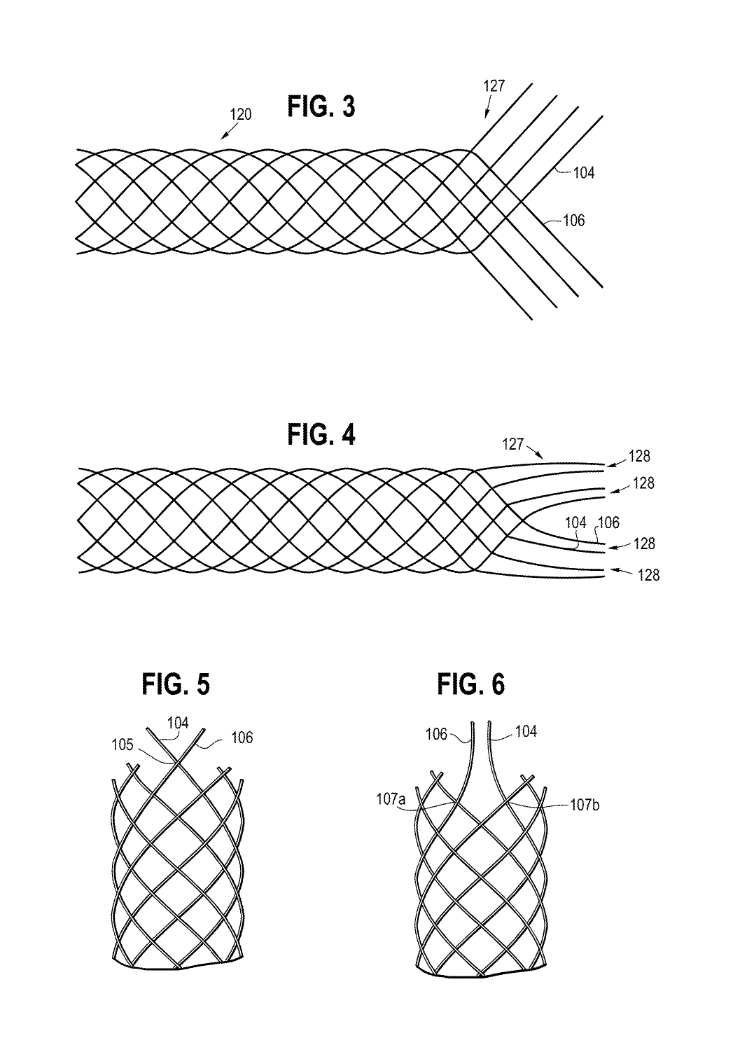

With reference to FIG. 3, once the mandrel is removed from the machine, a plurality of loose wire ends 127 may extend from at least one of the ends of the pre-woven stent 120. The loose wire ends 127 are the ends of the plurality of first and second wires 104, 106, and will become sharp ends 118 after being trimmed.

With reference to FIG. 4, the loose wire ends 127 may then be untangled and arranged in pairs 128. In particular, one of the first wires 104 and one of the second wires 106 may be paired together for being treated together to define a crown.

After being arranged in pairs, the loose wire ends 127 may be trimmed to define the sharp ends 118 that can then be subsequently treated to create the crowns 124. Prior to trimming the loose wire ends 127, a portion of the mandrel may be removed to provide access to the desired trim location. The mandrel will be discussed in further detail below.

For the purposes of discussion, the paired first wire 104 and second wire 106 may be joined together to eliminate the sharp ends 118. The wires 104 and 106 that are selected to be paired together and joined together can be any of the first wires 104 or second wires 106, as the woven stent includes multiple wires that may be classified as first wires 104 or second wires 106.

In one example, as shown in FIG. 5, wires that are paired together to be joined are circumferentially adjacent at the end to be treated to treat the sharp ends 118. Circumferentially adjacent wires may be defined as the two wires that extend from a common crossover point 105. The common cross-over point 105 is the cross-over point between the two wires that is closest to the end of the stent that is being treated, such that the portions of the wires 104 and 106 that extend axially beyond the cross-over point 105 do not cross over again after crossing over at the common cross-over point 105.

In another example, shown in FIG. 6, wires that are paired together to be joined are circumferentially adjacent, but may be defined as wires that extend from an adjacent pair of cross-over points 107a and 107b. Adjacent cross-over points 107a and 107b may also be cross-over points that are the points that are closest to the end of the stent to be treated, such that the wires do not cross-over again after crossing over at the adjacent cross-over points 107a and 107b.

Thus, the pairs of wires 104 and 106 that are joined together can be circumferentially adjacent whether they are joined from the common cross-over point 105 or the adjacent cross-over points 107a and 107b. Whether the wires are joined from the common cross-over point 105 or the adjacent cross-over points 107a and 107b will affect the shape of the paired wires 104 and 106 when they are joined.

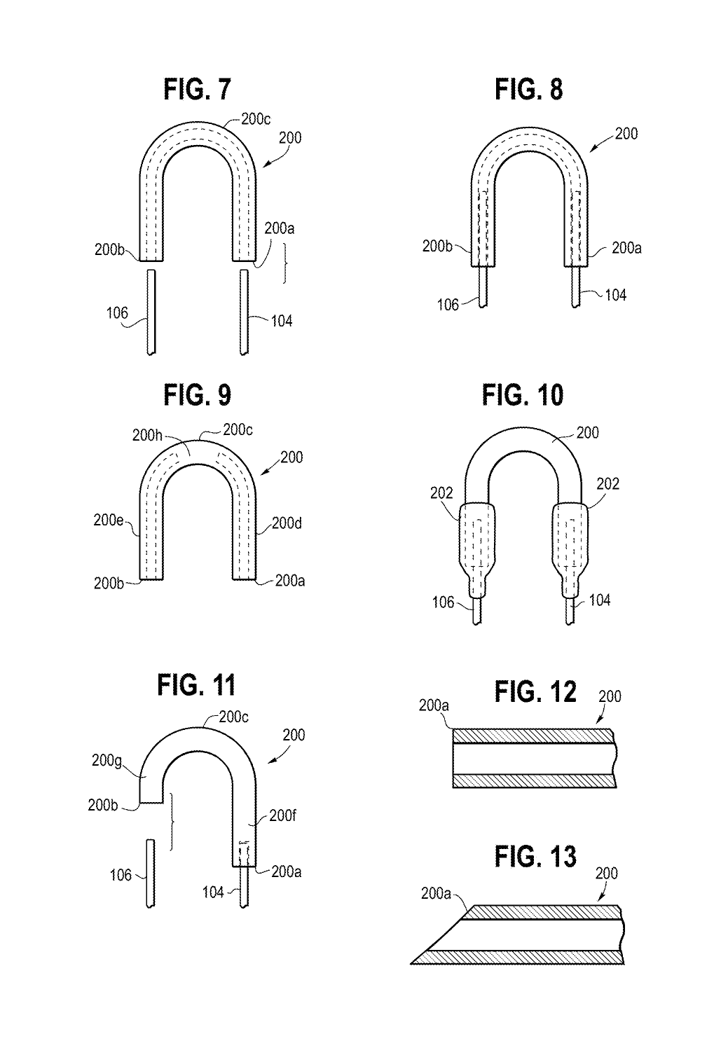

With reference to FIGS. 7 and 8, to join the wires, a cap 200 may be placed over the sharp ends 118 defined by the pair of wires 104 and 106. The cap 200 is separate piece from the woven stent and the wires 104 and 106. The cap 200 includes a first end 200a and a second end 200b. The cap 200 is preferably a unitary structure that extends continuously from the first end 200a to the second end 200b. The cap 200 extends from the first end 200a in a direction axially away from the end of the woven stent to be treated and toward a bend point 200c, where the cap 200 bends and extends back axially toward the second end 200b. The cap 200 may be described as being U-shaped or in the form of an inverted U-shape.

The cap 200 is configured such that the sharp ends 118 of the wires 104 and 106 to be joined will be received in respective ends of the cap 200. For example, the first wire 104 may be received in the first end 200a and the second wire 106 may be received in the second end 200b. Alternatively, the first wire 104 may be received in the second end 200b and the second wire 106 may be received in the first end 200a.

As the cap 200 is configured to receive the ends of the wires 104 and 106, the cap 200 may be in the form of a thin-walled cannula or ultra-thin walled cannula. Put another way, the cap 200 may include a lumen extending fully through the cap 200 from the first end 200a to the second end 200b. However, in another approach, the cap 200 may include separate lumens at the first end 200a and the second end 200b, with the area of the cap 200 at the bend point 200c being closed, as shown in FIG. 9. In either example, the first end 200a and the second end 200b are both open to receive the ends of the wires 104 and 106.

The cap 200 may be made from stainless steel, NiTi, PTFE, or other polymeric of metallic materials, such as polyethylene, ultra-high molecular weight polyethylene, polyester, nylon, or radiopaque materials such as tungsten, tantalum, molybdenum, platinum, gold, zirconium oxide, barium salt, bismuth salt, hafnium, and/or bismuth sub-carbonate.

The cap 200 is preferably fixed to the wires 104 and 106, or fixably attached to the wires 104 and 106. Fixed or fixably attached means an attachment method that substantially prevents the wires 104 and 106 from being removed from the cap 200 absent a significant force or cutting procedure, such that the cap 200 will remain attached and substantially immoveable relative to the wires 104 and 106 during normal operation and placement with the body.

The cap 200 may be attached in a variety of ways. In a preferred approach, the cap 200 is crimped to the wires 104 and 106. Crimping the cap 200 to the wires 104 and 106 may be performed by traditional crimping procedures, where the material of the cap 200 is compressed onto the wires 104 and 106 to create a friction fit between the cap 200 and the wires 104 and 106.

In another approach, the cap 200 may be attached to the wires 104 and 106 via bonding, such as via the use of adhesives. For example, an adhesive may be applied to the wires 104 and 106, which are inserted into the cap 200, or an adhesive may be inserted into the cap 200, and the wires 104 and 106 may inserted into the cap 200 and into contact with the adhesive.

In another approach, the wires 104 and 106 may be welded to the cap 200. The cap 200 may also be soldered to the wires 104 and 106.

The cap 200 may alternatively be attached to the wires 104 and 106 via a press-fit. In this approach, the wires 104 and 106 may have a thickness that is slightly larger than the nominal width of the openings at the ends 200a and 200b of the cap 200. Alternatively, the width of the openings of the cap 200 may have a slight taper such that the width reduces toward the interior of the lumen of the cap 200, such that insertion of the wires 104 and 106 creates a press-fit when the wires 104 and 106 are inserted into the cap 200.

In another approach, the cap 200 may be attached to the wires 104 and 106 via a membrane coating 202, as shown in FIG. 10. In this approach, the wires 104 and 106 are inserted into the cap 200, where the wires 104 and 106 may be slightly moveable relative to the cap 200. A membrane coating 202 may be applied over the intersection of the wires 104, 106 and the cap 200, thereby retaining the wires 104, 106 relative to the cap 200. The membrane coating may be applied over the entire cap 200, or may be applied over the entire woven stent after the caps 200 have been placed over the ends of the wires 104 and 106.

In one approach, the cap 200 includes a pair of legs 200d and 200e corresponding, respectively, to the first end 200a and second end 200b, such as in the cap shown in FIG. 9. The legs 200d and 200e may be approximately the same length extending from the bend point 200c to the ends 200a and 200b.

In another approach, the cap 200 may include legs 200f and 200g corresponding, respectively, to the first end 200a and second end 200b, where the legs 200f and 200g have different lengths extending from the bend point 200c. In this approach, it may be easier to insert the sharp ends 118 of the wires 104 and 106 sequentially into the ends of the cap 200 when the wires 104 and 106 have approximately the same length extending from their final cross-over point. The leg 200f may be longer than the leg 200g, or the leg 200g may be longer than the leg 200f.

The cap 200 with different leg lengths may also be used with wires 104 and 106 having different lengths. Similarly, the cap 200 with equal length legs may be used with wires 104 and 106 having different lengths or the same lengths.

The ends 200a and 200b may be flat, as shown in FIG. 12, such that the width of the opening corresponds to the width of the lumen within the cap. In another example, the ends 200a and 200b may be beveled, as shown in FIG. 13, such that the opening at the ends 200a and 200b is greater than the width of the lumen of the cap 200. The beveled form of the ends 200a and 200b may provide an easier insertion path for the wires 104 and 106. The ends 200a and 200b may alternatively be arranged such that one end is beveled and the other end is flat.

For purposes of discussion, the cap 200 will be described as having the legs 200d and 200e (equal length legs). However, it will be appreciated that discussion of the equal length legs may also be applied to the cap 200 with different length legs, unless otherwise noted.

The cap 200 has been described as having a U-shape. In this form, the legs 200d and 200e may extend substantially parallel to each other from the ends 200a and 200b toward the bend point 200c, as shown in FIGS. 7-11. The legs 200d and 200e will then curve toward the bend point 200c. In this case, the cap 200 may include a curved portion 200h having a radius. The cap 200 is therefore symmetrical relative to the bend point 200c for the cap 200 with equal length legs. The cap 200 with different length legs may be symmetrical up to the end of the shorter leg.

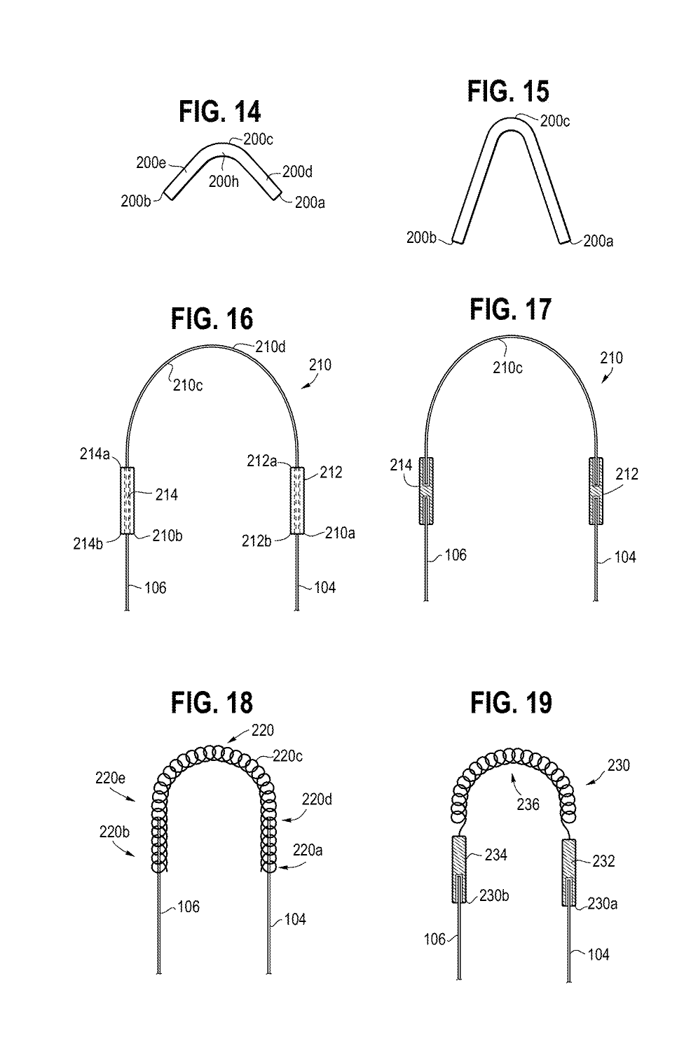

The cap 200 may also have a V-shaped form, as shown in FIG. 14. In the V-shaped form, the bend point 200c is not necessarily in the form of a sharp tip, of course. Rather, the legs extend from the bend point in a non-parallel fashion. Put another way, the legs extend away from the bend point 200c such that the distance between the legs increases up to the ends 200a and 200b. The cap 200 may also be described in this form as having a tapered leg shape. The V-shaped cap 200 may also be considered to be symmetrical relative to the bend point 200c, with the legs 200d and 200e curving toward the bend point 200c and having the curved portion 200h.

The cap 200 may also be non-symmetrical relative to the bend point 200c, as shown in FIG. 15. In this approach, one of the legs of the cap 200 may be formed similar to the U-shaped cap 200 relative to the bend point 200c, with the other leg having a shape similar to the V-shaped cap 200. Thus, the curved portion 200h is longer on the side of the V-shaped leg.

The above described caps 200 may be used to treat multiple pairs of wires 104 and 106 around the end of the woven stent. The same style or shape of cap 200 may be used at each wire pair, or different styles of the cap 200 may be used at different wire pairs.

With reference to FIG. 16, in another example, a bridge cap 210 may be used to treat the sharp ends 118 of the wires 104 and 106. The bridge cap 210 differs from the cap 200 described above in that the curved portion and bend point may be a different material or shape from the ends of the cap, as further described below.

The bridge cap 210 may include a first end 210a and a second end 210b, with the first end 210a and the second end 210b being parts of separate pieces joined by a curved bridge portion 210c. The curved bridge portion 210c includes a bend point 210d. The first end 210a may be part of first leg 212 having an inner end 212a and an outer end 212b, with the outer end 212b defining the first end 210a of the bridge cap 210. The second end 210b may be part of a second leg 214 having an inner end 214a and an outer end 214b, with the outer end 214b defining the second end 210b of the bridge cap 210.

The first leg 212 and second leg 214 may be constructed similarly to the above described cap 200, such that the first leg 212 and second leg 214 may be in the form of thin-walled or ultrathin-walled cannulas. Thus, the first and second legs 212, 214 may define a lumen extending fully therethrough.

The inner ends 212a and 214a of the legs 212, 214 may receive the ends of the curved bridge portion 210c in a manner similar to that described above with respect to the cap 200 receiving the wires 104 and 106. Thus, the curved bridge portion 210c is received in the ends of the legs 212 and 214 and fixed in place and fixedly held. The curved bridge portion 210c may be fixed via crimping, bonding, welding, soldering, membrane covering, press-fit, or other method for attaching a wire in place relative to a cannula.

With the curved portion 210c being fixed to the legs 212 and 214, the bridge cap 210 is therefore defined and capable of receiving the sharp ends 118 of the wires 104 and 106 in a manner similar to the cap 200, with the same types of attachment mentioned above with respect to the cap 200.

The curved portion 210c may be in the form of a solid wire. The curved portion 210c may also be in the form of a braided wire, providing increased flexibility and softness in some instances. The bridge portion 210c may be made of a radiopaque material. The bridge portion 210c may also be in the form of a suture material for increased flexibility and softness.

The bridge portion 210c, as well as the legs 212 and 214, may be made from the same materials described above for the cap 200.

The legs 212 and 214 may include flat ends or beveled ends, similar to those described above with respect to the cap 200. The flat ends or beveled ends may be disposed at the ends that received the wire 104/106 and/or the ends that receive the bridge portion 210c.

The bridge cap 210 may have increased flexibility and softness relative to the cap 200. In one approach, the bridge cap 210, via the curved portion 210c, may be biased into its curved shape to define a U-shape or V-shape similar to those described above. In another approach, the bridge cap 210 may be biased toward a straight shape but sufficiently flexible to be bend into the curved form to receive the ends of the wires 104 and 106. In another form, the bridge cap 210 may be generally un-biased to any particular shape and flexible to be joined to the ends of the wires 104 and 106. As illustrated in FIG. 16, when installed, the bridge cap 210 may have a curved shape, whether due to a bias or after being bent from a straight shape after receiving the wires 104 and 106.

The legs 212 and 214 have been described as thin-walled cannulas, but the legs 212 and 214 may also be in the form of a solid-core rod having openings at each end, but without a lumen extending fully therethrough, such that the middle of the legs 212 and 214 is closed, as shown in FIG. 17. In this approach, insertion of either the wires 104, 106 or the curved bridge portion 210c will be stopped by the closed middle of the legs 212 and 214. Either or both of the legs may be thin-walled cannulas or solid-core rods.

The bridge cap 210 may be used along with the cap 200 to treat the ends of the same woven stent, or the bridge cap may be used to treat all the wire pairs of the woven stent. Different types of the bridge cap 210 may be used on the same woven stent, or the same type of bridge cap 210 may be used.

With reference to FIG. 18, in yet another approach, an alternative type of cap in the form of a coil cap 220 may be used to treat the sharp ends 118 of the wires 104 and 106. The coil cap 220 may have a shape similar to the cap 200 or bridge cap 210 described above. In the case of the coil cap 220, the coil cap 220 is defined by a bent or curved micro-coil rather than the cannula or bridged legs described above.

The coil cap 220 includes first and second open ends 220a and 220b. The coil cap 220 is defined by a plurality of wire loops extending in a helical manner from the first end 220a to the second ends 220b. The wire loops of the coil cap 220 define a lumen extending from the first end 220a to the second end 220b.

The coil cap 220 is similar to the cap 200 in that it is a generally unitary structure extending from the first end 220a to the second end 220b and defines a lumen extending therebetween. The coil cap 220 differs from the cap 200 in that it may have openings along its length between the adjacent wire loops that defines the coil shape.

The coil cap 220 may also have increased flexibility and softness relative to the thin walled cannula style of the cap 200. The coil cap 220 may be formed such that it is biased to a shape resembling the shapes described above of the cap 200, such as a U-shape or V-shape, and with different leg lengths or similar leg lengths. The coil cap 220 may alternatively be biased into a straight coil form that is bendable to create a U-shape and join the ends of the wires 104 and 106.

The coil cap 220, when bent either during attachment to the wires 104 and 106 or biased to a bent shape, may similarly include a bend point 220c and leg portions 220d and 220e. The leg portions 200d and 200e may curve toward the bend point 220c.

The plurality of loops that define the coil cap 220 may be looped or coiled such that adjacent loops are in contact with each other when the coil cap 220 is straight, and may expand away from each other at the location of bending or curving, with the outer portion of the bend having gaps between the loops and the inner portion of the bend having the loops in contact with each other.

Alternatively, the plurality of loops that define the coil cap may be looped or coiled such that there are gaps between each of the loops, even when straight. In another form, some of the loops may contact each other, while others are looped to have a gap therebetween.

The coil cap 220 may be made from the any of the materials described above with respect to the cap 200, such as stainless steel, NiTi, etc.

The coil cap 220 may be attached to the wires 104 and 106 in a manner similar to that described above with respect to the cap 200, such as soldering, welding, crimping, etc. In the case of a press-fit, the coil cap 220 may be coiled to have a tapered lumen or a smaller lumen width relative to the wires 104, 106, such that the loops will be spread outward and the bias in the coil will exert a radially inward force on the wires 104 and 106 to hold them in place. However, it may be preferable to supplement this type of press-fit attachment with and adhesive or other attachment method described above to limit instances where the coil could flex and loosen relative to the wires 104 and 106.

The coil cap 220 may in the form of a solid wire that is looped into the coil-form, or it may be in the form of a braided wire looped into the coil-form.

In another embodiment, illustrated in FIG. 19, a coil bridge cap 230 may include a first end 230a and a second end 230b, and may be used to join the ends of the wires 104 and 106. The coil bridge cap 230 may be constructed similar to the bridge cap 210 described above, and may include legs 232 and 234 similar to the legs 212 and 214. The legs 232 and 234 may therefore be in the form of cannulas with open inner and outer ends.

In this example, the coil-bridge cap 230 includes a curved portion 236 that extends between the inner ends of the legs 232 and 234. The curved portion 236 is in the form a microcoil, similar to the coil cap 220 described above. The description of the coil cap 220 may be equally applied to the curved portion 236 of the coil-bridge cap 230. For example, the coiled curved portion 236 may be biased to a U-shape or V-shape, or may be biased to a straight shape and be flexible and bendable.

In the coil-bridge cap 230, the wires 104 and 106 are not received in the coiled curved portion 236. Rather, the wires 104 and 106 are received in the legs 232 and 234 in a manner similar to the bridge cap 210 described above. The coiled curved portion 236 may be attached to the legs 232 and 234 in a manner similar to the description above regarding the bridge cap 210, where the ends of the coiled curved portion 236 are received in the inner ends of the legs 232 and 234, and fixed in place via crimping, welding, bonding, soldering, etc.

In another approach, the legs 232 and 234 may be attached to the coiled curved portion 236 in a manner similar to the wires 104 and 106 being attached to the coil cap 220. In this approach, the inner ends of the legs 232 and 234 are received within the end openings of the coiled curved portion 236. With the inner ends of the legs within the ends of the coiled curved portion 236, the legs 232 and 234 may be fixed to the coiled curved portion 236 in a manner described above, such as crimping, bonding, welding, soldering, etc.

The coiled curved portion 236 may be made from a solid wire or braided wire looped into the coil-form. The coiled curved portion 236, along with the leg portion 232 and 234, may be made from the same materials described above, such as stainless steel, NiTi, radiopaque material, etc.

The use of a coiled curved portion 236 in the coil-bridge cap 230 may allow for increased flexibility, softness, and radiopacity relative to the other caps described above.

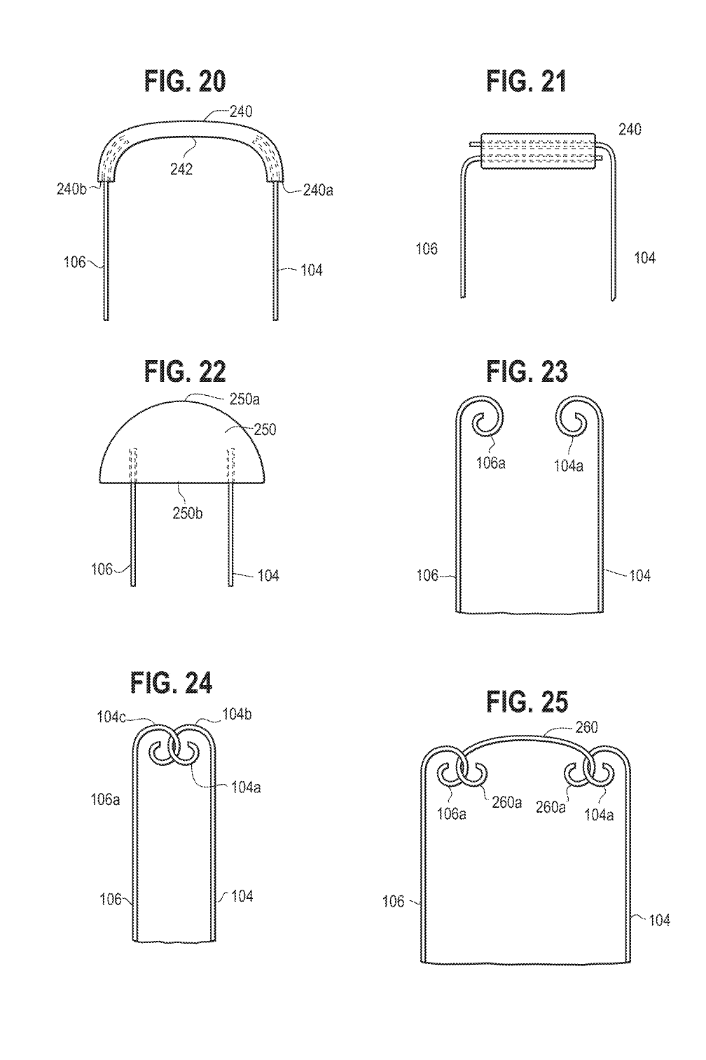

With reference to FIG. 20, in yet another embodiment, the sharp ends 118 of the wires 104 and 106 may be treated using a soft cap 240. The soft cap 240 may in the form a soft and flexible tube with open ends 240a and 240b. The soft cap 240 may be in the form of heat shrink material, or a soft thin-walled rubber or polymer.

The soft cap 240 may have an elongate middle portion 242 extending between the ends 240a and 240b. The ends 240a and 240b may be in the form of extensions from the middle portion 242, or may be in the form of bent ends, where the ends 240a and 240b bend toward the wires to be treated.

The soft cap 240 may define a lumen extending fully through the soft cap 240 between the ends 240a and 240b. Thus, the soft cap 240, like the caps described above, may receive the ends of the wires 104 and 106 in the openings at the ends of the soft cap 240.

In one approach, when the using the soft cap 240, the wires 104 and 106 may be bent at their sharp ends 118. The bend at the ends of the wires 104 and 106 may be slight. The slight bends at the end of the wires 104 and 106 will substantially limit instances of the wires 104 and 106 poking through the material of the soft cap 240 when the wires are inserted into the soft cap 240.

The soft cap 240 is preferably attached and fixed to the wires 240 using a heat shrink process, where the soft cap 240 is heated, causing the soft cap 240 to radially compress around the ends of the wires 240 that are disposed within the soft cap 240. At the conclusion of the heat shrinking process, the wires 104 and 106 are held in place relative to the soft cap 240, and the sharp ends of the wires 104 and 106 are disposed within the soft cap 240.

In another approach, shown in FIG. 21, with the soft cap 240, the wires 104 and 106 may be bent more substantially, such that the wires 104 and 106 extend toward each other and overlap. In this approach, the soft cap 240 may be substantially straight when the wires are inserted. With the soft cap 240 being a heat shrink material, the opening or lumen through the soft cap may be large enough relative to the wires 104 and 106 that both wires 104 and 106 may extend into or through the soft cap 240 into an overlapping arrangement.

In this approach, the soft cap 240 may be heated, such that the soft cap 240 will shrink around the overlapping wires 104 and 106. The wires 104 and 106 may extend fully through the soft cap 240, such that the ends are exposed out of the soft cap 240.

With the soft cap 240 surrounding the wires 104 and 106, the sift cap and wires may be further bent or manipulated to define a curved crown type shape, if desired.

In another example, a molded cap 250 may be used. The molded cap 250 may have a bulbous or curved outer end 250a and a receiving end 250b, with the receiving end 250b being configured to receive the ends of the wires 104 and 106 to be treated.

The molded cap could be a solid material, with the wires 104 and 106 received in openings at the receiving end 250b and bonded or welding or press-fit to the molded cap 250. The molded cap 250 may also be a flexible material, with the ends of the wires 104 and 106 penetrating the receiving end and being held in place via press-fit, bonding, welding, or the like. The molded cap 250 may also be crimped to the wires 104 and 106, depending on the material and thickness and the molded cap 250. The molded cap 250 may be made from any of the above described materials.

In another example, shown in FIGS. 23 and 24, the wires 104 and 106 may be paired together without a “cap” where the wires are received in an opening. In this approach, the wires 104 and 106 may be treated such that the wires 104 and 106 are bent into a curled end having a tail 104a and 106a, respectively. In this approach, the wires 104 and 106 may be joined together by clipping together the tails 104a and 106a together, where the tails 104a and 106a may be twisted or crimped together to hold them in place. In this approach, the axially outermost point of the wires 104 and 106 is outboard from the interface between the tails 104a and 106a, and defines a pair of bumps 104b and 106b.

In another approach, shown in FIG. 25, the tails 104a and 106a of the curled ends of the wires 104 and 106 may be joined by a separate link 260. The link 260 may be in the form of a solid wire or a woven wire, or a suture, and may include first and second ends 260a and 260b. The ends 260a and 260b are curled similar to the ends of the wires 104 and 106, and the curled ends 104a and 106a are joined together by their respective curls with the link 260. The curls of the link 260 and the wires 104 and 106 may be twisted or otherwise fixed to each other via crimping or bonding or the like.

Each of the above described caps may be used for each wire pair of the woven stent to be paired, or the woven stent may include different caps for different wire pairs. For purposes of discussion involving the use of the various caps, the cap 200 will be referenced.

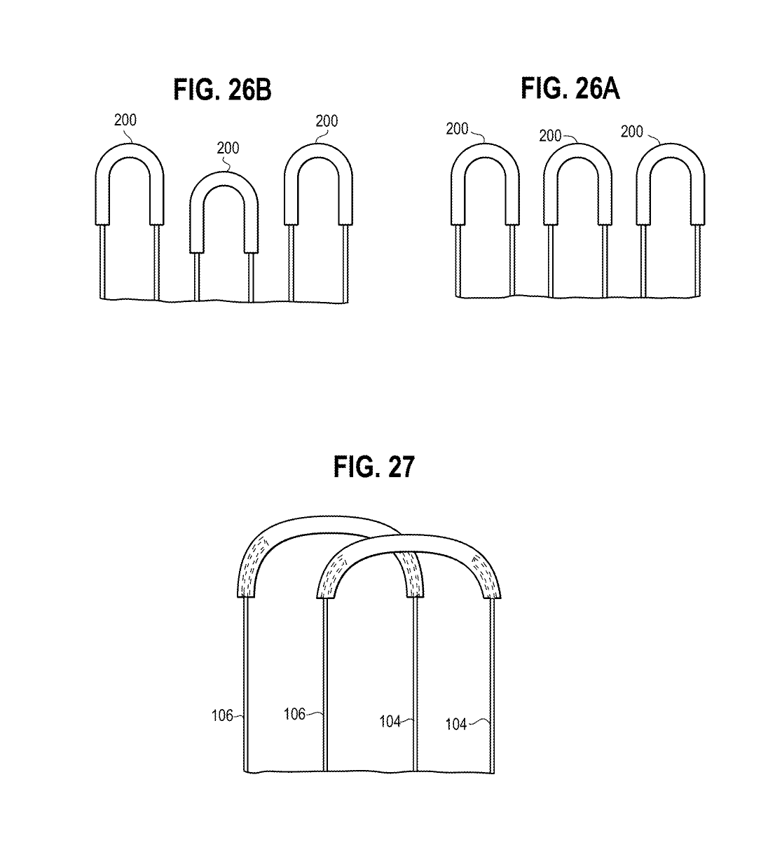

There are, of course, multiple pairs of wires 104 and 106 circumferentially around the end of the stent. The cap 200 may be thicker or more robust that the ends of the wires 104 and 106. The caps 200 do not have to all be located at the same axial location, such that the crowns defined by the caps are at the same axial point. In one form, shown in FIG. 26A, the caps 200 are indeed disposed at the same approximate axial point relative to each other, taking into account minor assembly and manufacturing tolerances.

In another approach, shown in FIG. 26B, the caps 200 are specifically arranged relative to each other in a staggered arrangement, such as in an alternating arrangement. In this form, every other cap 200 is located at a first axial location, with alternating caps 200 being disposed at a second axial location. The caps 200 may also be arranged at more than two axial locations, such as a portion of the caps 200 being arranged at a third axial location, a fourth axial location, etc. To control the axial location of each cap 200, the pairs of wires 104 and 106 to be capped can be cut at different lengths.

The wires 104 and 106 to be paired and capped have been previously described as being circumferentially adjacent at the end to be treated. Alternatively, a wire 104 may be paired with a wire 106 that is circumferentially further away than the adjacent wire 106. In this approach, the caps 200 could overlap each other in some cases, as shown in FIG. 27. In yet another approach, a pair of wires 104 could be paired with each other and capped, with a pair of wires 106 being paired with each other and capped. It will be appreciated that various pairings of the wires 104/106 can be joined together via one of the caps described above.

Unlike prior designs where the treated ends of machine woven stents are attached to each other and welded or bonded directly together, the use of the cap 200 provides a separate structure that will absorb the stress and strain on the end of the stent. This construction thereby reduces instances of the treated ends of the stents breaking and creating sharp ends and potential unraveling of the finished stent.

Having described the various types of caps, the process for treating the ends of the pre-woven stent 120 will now be described in further detail. The described process is preferably application to pre-woven stents that are machine woven, but the caps could also be used on the ends of stents that were previously handwoven, or stents that have been damaged and requiring treating to remove sharp ends. For purposes of discussion, the procedure for treating the pre-woven stent 120 that is a machine woven stent will be described, and the pre-woven stent 120 will be referred to as machine woven.

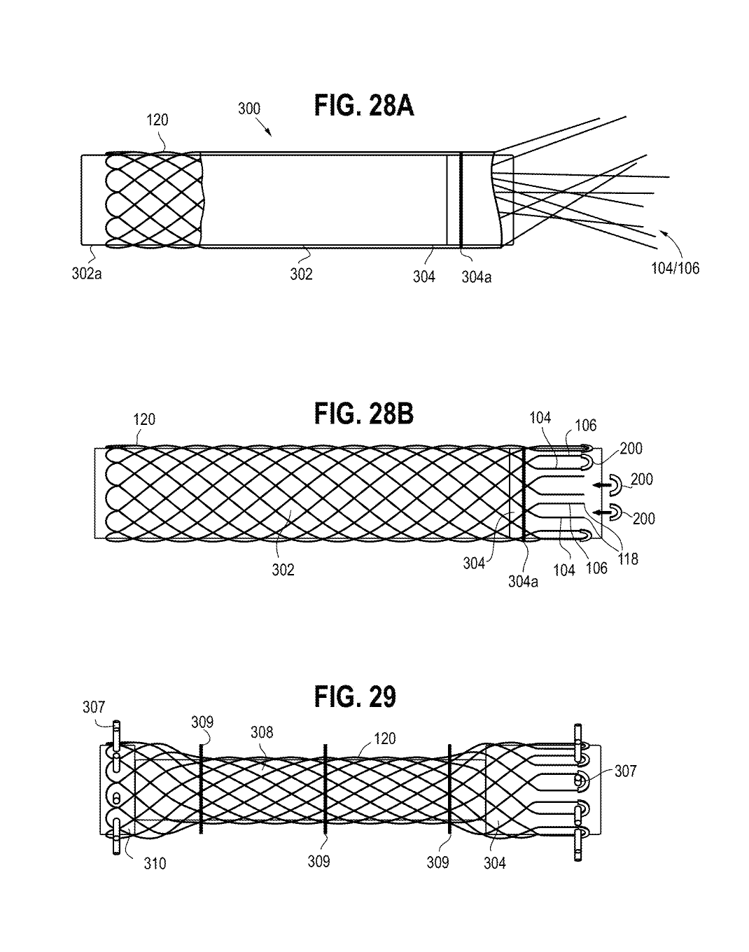

With reference to FIG. 28A, the machine woven stent 120 is machine woven on a mandrel 300 having a generally constant diameter, thereby creating a “parallel” stent shape, where the resulting machine woven stent 120 has a generally constant diameter corresponding to the diameter of the mandrel 300. The mandrel 300 may have multiple portions, including a body portion 302 and a head portion 304. The head portion 304 is disposed axially adjacent the body portion 302. Each of these portions are separable relative to each other and removable, but in practice it is preferable to remove the body portion 302 and leave the head portion 304 in place during the process for creating a flanged stent. These portions could also be referred to as first and second portions.

The body portion 302 has a base end 302a, where the wires of the machine woven stent 120 are initially disposed, such that the end of the machine woven stent 120 corresponding to the base end 302a may be considered “closed” and may not require treating. The machine woven stent 120 is woven on the mandrel in a manner known in the art, leaving a plurality of loose wires 104 and 106 extending beyond the head portion 304. Ligatures 304a may be applied at the head portion 304 of the stent 120 while it is still on the mandrel 300 to prevent unraveling of the wires 104 and 106 after weaving.

The wires 104 and 106 may be untangled or unwoven beyond the head portion 304 and arranged in in the desired pairs (shown in FIG. 4). In a preferred form, the adjacent wires 104 and 106 are arranged in pairs.

As shown in FIG. 28B, the wires 104 and 106 may then be trimmed in pairs, individually, or in another group, with the wires 104 and 106 being trimmed to the desired length relative to the head portion 304. Trimming the wires 104 and 106 defines the sharp ends 118 of the wires to be treated by providing the cap 200. The cap 200 is applied to the sharps ends 118 of the pairs of wires 104, 106. The cap 200 is then secured to the wires 104, 106 in a manner described above, for example via crimping. Each wire pair may be capped with the cap 200, at the same axial location or at a different axial location from other caps 200.

Once each of the pairs of wires 104 and 106 has been capped, the machine woven stent 120 may be considered treated, because the machine woven stent 120 no longer includes any exposed sharp ends. The resulting stent has a generally constant diameter and may be considered a parallel stent. The remainder of the mandrel 300 may be removed entirely, resulting in a stent with a constant diameter after heat setting.

In an alternative approach, shown in FIG. 29, a flanged stent may be formed via additional steps. In particular, the body portion 302 of the mandrel 300 may be removed, leaving the head portion 304 in place. Prior to removing the body portion 302, the treated ends of the stent may be pinned or otherwise secured to the head portion 304. One type of securement is through the use of radially extending pins 307 (FIGS. 29 and 31) that extend radially outward from the head portion 304, such that the pins 307 extends outward through the caps 200 or the crowns of the closed end of the stent. After the body portion 302 is removed, the pins 307 will keep the treated end of the stent secured to the head portion 304. A reduced diameter portion 308 may then be inserted in place of the body portion 302, such that the head portion 304 has a larger diameter than the reduced diameter portion 308.

The reduced diameter portion 308 may have a constant diameter, or it may include a tail portion 310 having a greater diameter than the remainder of the reduced diameter portion 308. The tail portion 310 may have a diameter similar to the head portion 304. The reduced diameter portion 308 may be inserted into the stent 120 and into attachment with the head portion 304. Ligatures 309 may be disposed around the stent after the reduced diameter portion 308 has bene inserted into the stent. The ligatures 309 may be placed at the interface between the reduced diameter portion 308 and the larger diameter head portion 304. Pins 307 or another securement may be used to secure the opposite end of the stent after the reduced diameter portion 308 has been inserted.

The stent 120 may then be heat treated, where the middle portion of the stent has the reduced diameter middle of the reduced diameter portion 308, and the end of the stent at the head portion 304 remains at a larger diameter. The result of this heat setting is a flanged or stepped stent. The stent 120 may be flanged or stepped at both ends (in the case of a head set mandrel 308 with a tail portion 310), or it may include a single flanged end in the case of a single diameter reduced diameter portion 308. The tail portion 310 may also be a separate piece that is placed over the reduced diameter portion 308, resulting in a dual flanged version. It will be appreciated that various stepped shapes could be created using different shapes and pieces of a reduced diameter portion.

In the above described approach, the head portion 304 and tail portion 310 may include pins or other structure that extend through the ends of the stent 120 to keep the ends in place. In the case of a separate tail section 310, which may be considered the preferred approach, the tail section 310 is preferably inserted into the end of the stent 120 prior to inserting the reduced diameter portion 308.

The machine weaving process cannot create a stepped stent. The above described process allows for the use of machine weaving for a stent with treated ends and flanged shape that can be produced in a manner of minutes as opposed to a manner of hours for hand weaving a flanged stent.

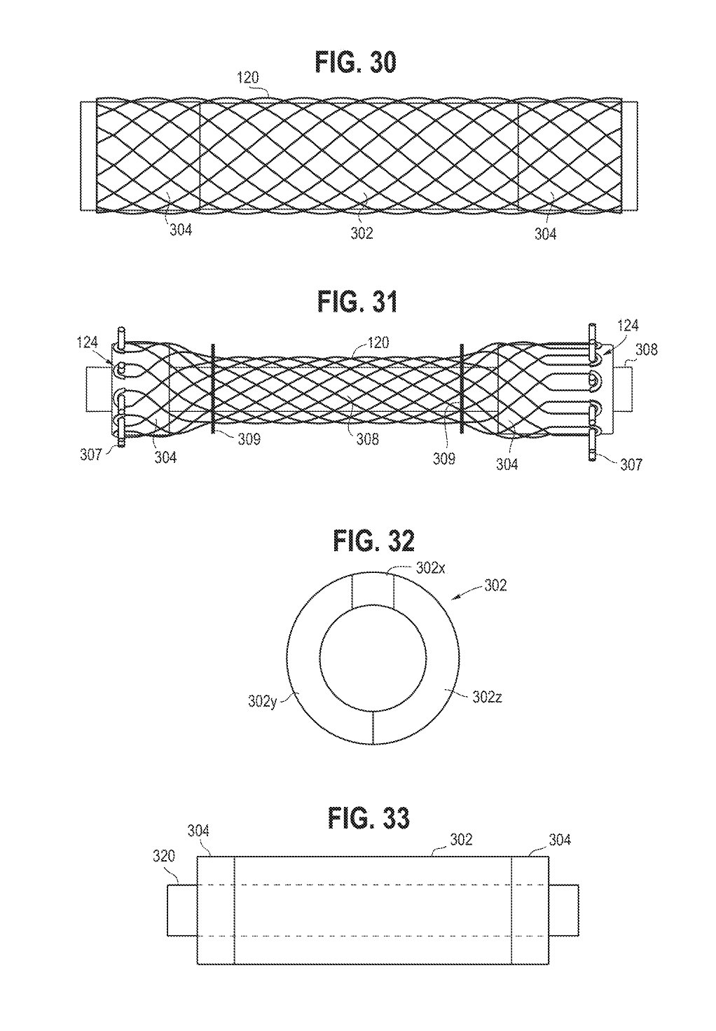

The above described process may also be used for machine woven stents 120 without a closed end. In this approach, shown in FIGS. 30 and 31, the mandrel 300 may include the body portion 302, and two head portions 304 at opposite ends. In this approach, the above described process of pairing, trimming, and pinning the ends of the stent is performed at each opposite end of the stent 120.

In this approach, however, the body portion 302 cannot be simply removed from the end of the stent 120, because there is a head portion 304 at each end. As described above, it is desirable for the head portions 304 to remain engaged with the capped ends of the stent until after heat setting has occurred.

Thus, in this approach, the body portion 302 is collapsible, such that it may be removed out through one of the head portions 304 after being collapsed. The body portion 302 is collapsed and removed after the ends of the stent 302 have been paired, trimmed, capped, and pinned to the head portions 304. Once the body portion 302 is collapsed and removed, only the head portions 304 remain, with the ends of the stent 120 being secured to the head portions 304. The area between the head portions 304 is empty, aside from the middle of the stent.

After removing the body 302, the reduced diameter portion 308 is inserted through one of the head portions 304, and secured to the head portions 304, and the heat setting process may be performed. Similar to the above, ligatures 309 may be disposed to compress the stent onto the reduced diameter portion 308.

With reference to FIG. 32, the body portion 302 and the head portions 304 are generally tubular. For example, they may have an outer diameter of about 25 mm, with a wall thickness of about 2 mm. FIG. 32 illustrates an example of the body portion 302 being collapsible. In this approach, the body portion 302 may include three sections 302x, 302y, and 302z. To collapse the body portion 302, section 302x may collapse inward first, allowing sections 302y and 302z to collapse toward each other. With the body portion 302 collapsed into these sections, it may be removed through the tubular shape of the head portion 304. It will be appreciated that the collapsible body portion could have more than three sections.

With reference to FIG. 33, a core pin 320 extends through the collapsible body portion 302 and the head portions 304. The core pin 320 has an outer diameter that generally corresponds to the inner diameter of the collapsible body portion 302, such that the body portion 302 will remain in its tubular shape while the core pin 320 is inserted therethrough. Withdrawing the core pin 320 will allow the body portion 304 to collapse into the space vacated by the core pin 320.

The above described reduced diameter portion 308 may have a diameter of about 21 mm to go along with the example of the 25 mm sizing described above. It will be appreciated that other relative sizes may also be used depending on the desired size of the stent and the flanges created.

Preferably, the reduced diameter portion 308 has a length that is greater than or equal to the length between the outer ends of the head portions 304. FIG. 31 illustrates the reduced diameter portion 308 being longer that the length between the ends of the head portions 304, such that it extends beyond the head portions after sliding into the head portions 304 and the stent 120.

The ligatures 309 may be positioned close to the inner ends of the head portions 304 to create a sharp shoulder in the flanged stent. The ligatures 309 are shown spaced away from the head portions 304 for illustrative purposes, but it will be appreciated that the ligatures 309 could be placed against the head portions 304 to create a sharper shoulder than illustrated. The ligatures 309 may be in the form of NiTi wire.

At the conclusion of the heat setting process, the various pieces of the mandrel 300 may be removed from the stent 120, leaving in place the flanged shape. The ligatures 309 and 307 are also removed after heat setting. The result of this heat setting process is a fully formed stent with capped ends having flanges at each end. The stent 120 is shape-set and self expanding.

The above described process allows the flanged shape to be created without having to increase the diameter of the ends of the stent after they have been capped. Rather, the middle portion of the stent is compressed inward to create the flanges. This is desirable because the middle portion of the stent is more stable than the ends of the stent that have been capped. The treated ends of the stent can be susceptible to losing their shape prior to heat setting, so it is advantageous to leave the treated and capped ends of the stent in their nominal shape, and alter the middle portion of the stent instead. Accordingly, it is desirable to perform the machine weaving process at a diameter of the desired diameter of the flanges. The collapsible body portion 302 allows for the treated ends to remain generally undisturbed. Without the collapsible body portion, one of the head portions 304 would have to be removed, leaving the treated end of the stent susceptible to losing its shape.

Thus, both ends of the stent 120 may be flanged without requiring one end to be closed in the machine weaving process. This approach allows the manufacture to be performed without having to reload the machine mandrel each time to machine weave the stent 120. In this approach, an extended length of machine woven stent may be created, with the machine woven stent 120 being cut at different locations to create multiple stents in a single machine weaving process.

The above described caps can be pre-formed and assembled prior to placement on the machine woven stent 120. The process of applying the caps may be automated. The machine stent 120 treated with the cap 200 (or other described caps) may be easily loaded within an introducer. The caps can allow for a tailored flexibility or softness depending on the needs of the patient, for example a soft crown like the coiled cap may be provided and can provide increased patient comfort. Of course, each of the above described caps provide increased comfort relative to sharp ends, and the caps provide added resistance to breaking that may occur from stresses applied at the ends of the stents.

A stent according to the present disclosure may have any suitable braid angle. The radial force of the stent may be controlled by adjusting the braid angle accordingly. Stents with higher braid angles typically exert greater radial force and exhibit greater foreshortening during expansion from a compressed state. Stents with lower braid angles typically exert lower radial force and experience less foreshortening upon expansion. In some instances, if the stent is partially or fully covered with a membrane material, the braid angle can be lowered because membrane coverings typically add rigidity to the stent structure. In addition to adjusting the braid angle, the radial force of the stent can be adjusted through selection of particular materials as well as the shape and size of the filaments or wires forming the stent structure.

Although the illustrated embodiments depict a stent having a central body portion and a flange, other stent configurations are possible. For example, a stent may include a double flange, two asymmetrically shaped flanges, or may entirely lack flanges and instead have a uniform or substantially uniform diameter along the entire length of the stent. A stent may include a uniform diameter along the length of the stent but include slightly flared proximal and/or distal ends. The central body portion may smoothly transition to a flange or flare, or alternatively, may progressively step up in diameter to a flange or flare. Generally, a stent may be implanted in a vessel (e.g., esophagus, duodenum, colon, trachea, or the like) such that the central body portion engages a diseased area and the flanges or ends engage healthy tissue adjacent the diseased area. Preferably, the flanges are configured to anchor the stent at the site of implantation, thereby reducing the incidence of proximal and distal migration. Preferably, the flanges are sized and shaped to accommodate the vessel or organ of implantation. For example, stents destined for lower esophageal implantation may have differently shaped and sized flanges compared to a stent designed for upper esophageal implantation. In addition to the soft crowns, the flanges may include other features or configurations designed to reduce incidence of tissue perforation and overgrowth. For example, the ends (e.g., the crown cells) of the flanges may curve or bend inward toward the stent lumen to minimize tissue damage at or near the stent ends. In certain embodiments, a stent may include other design elements configured to secure the stent at the site of implantation. For example, in certain embodiments, a stent may include small anchors, clips, hooks, or barbs that will anchor the stent to the internal wall of the targeted body lumen. In other embodiments, the stent may be sutured to the site of implantation at one or more portions of the stent structure.

A stent including treated ends may include one or more components configured to aid in visualization and/or adjustment of the stent during implantation, repositioning, or retrieval. For example, a stent may include one or more radiopaque markers configured to provide for fluoroscopic visualization for accurate deployment and positioning. Radiopaque markers may be affixed (e.g., by welding, gluing, suturing, or the like) at or near the ends of the stent at a cross point of filament(s) in the braid pattern. In certain embodiments, a stent may include four radiopaque markers with two markers affixed to a first flange and two to a second flange. Optionally, radiopacity can be added to a stent through coating processes such as sputtering, plating, or co-drawing gold or similar heavy metals onto the stent. Radiopacity can also be included by alloy addition. Radiopaque materials and markers may be comprised of any suitable biocompatible materials, such as tungsten, tantalum, molybdenum, platinum, gold, zirconium oxide, barium salt, bismuth salt, hafnium, and/or bismuth subcarbonate.

A stent including treated ends may include one or more loops, lassos, or sutures on the stent structure to facilitate repositioning or removal of the stent during or after implantation. For example, a stent may include a loop at or near the proximal end of the stent. The loop material may circumscribe the flange and in certain embodiments may be wound through the absolute end cells to affix the loop to the stent. The loop may comprise any appropriate biocompatible material, such as for example, suture materials or other polymeric or metallic materials such as polyethylene, ultra-high molecular weight polyethylene, polyester, nylon, stainless steel, nitinol, or the like. Optionally, the lasso may be coated with a material, such as polytetrafluoroethylene, to reduce frictional interactions of the lasso with surrounding tissue.

Stents including treated ends may be self-expanding, mechanically expandable, or a combination thereof. Self-expanding stents may be self-expanding under their inherent resilience or may be heat activated wherein the stent self-expands upon reaching a predetermined temperature or range of temperatures. One advantage of self-expanding stents is that traumas from external sources or natural changes in the shape of a body lumen do not permanently deform the stent. Thus, self-expanding stents may be preferred for use in vessels that are subject to changes in shape and/or changes in position, such as those of the peripheral and gastrointestinal systems. Peripheral vessels regularly change shape as the vessels experience trauma from external sources (e.g., impacts to arms, legs, etc.); and many gastrointestinal vessels naturally change shape as peristaltic motion advances food through the digestive tract. One common procedure for implanting a self-expanding stent involves a two-step process. First, if necessary, the diseased vessel may be dilated with a balloon or other device. The stent may be loaded within a sheath that retains the stent in a compressed state for delivery to the targeted vessel. The stent may then be guided to the target anatomy via a delivery catheter and thereafter released by retracting or removing the retaining sheath. Once released from the sheath, the stent may radially expand until it contacts and presses against the vessel wall. In some procedures, self-expanding stents may be delivered with the assistance of an endoscope and/or a fluoroscope. An endoscope provides visualization as well as working channels through which devices and instruments may be delivered to the site of implantation. A fluoroscope also provides visualization of the patient anatomy to aid in placement of an implantable device, particularly in the gastrointestinal system.

Mechanically expandable stents (e.g., balloon expandable stents) having treated ends may be made from plastically deformable materials (e.g., 316L stainless steel). A balloon-expandable stent may be crimped and delivered in a reduced diameter and thereafter expanded to a precise expanded diameter. Balloon expandable stents can be used to treat stenosed coronary arteries, among other vessels. One common procedure for implanting a balloon expandable stent involves mounting the stent circumferentially on a balloon-tipped catheter and threading the catheter through a vessel passageway to the target area. Once the balloon is positioned at the targeted area, the balloon may be inflated to dilate the vessel and radially expand the stent. The balloon may then be deflated and removed from the passageway.

Expandable stents according to the present disclosure may be formed by any suitable method as is known in the art. In certain embodiments, the expandable stents may be fabricated by braiding, weaving, knitting, crocheting, welding, suturing, or otherwise machining together one or more filaments or wires into a tubular frame. Such stents may be referred to as braided, woven, or mesh stents. A braided stent may be fabricated by, for example, use of a braiding mandrel having specifically designed features (e.g., grooves and detents) for creating such a stent. A variety of braiding patterns are possible, such as for example, one-under and one-over patterns or two-under and two-over patterns. The filaments or wires may be of various cross-sectional shapes. For example, the filaments or wires may be flat in shape or may have a circular-shaped cross-section. The filaments or wires may have any suitable diameter, such as for example, from about 0.10 to about 0.30 mm. As will be described in greater detail below, the expandable stents may formed from a variety of biocompatible materials. For example, the filaments or wires may comprise one or more elastically deformable materials such as shape memory alloys (e.g., 304 stainless steel, nitinol, and the like).

A stent including treated ends may be made from any suitable biocompatible material(s). For example, a stent may include materials such as stainless steel, nitinol, MP35N, gold, tantalum, platinum or platinum iridium, niobium, tungsten, iconel, ceramic, nickel, titanium, stainless steel/titanium composite, cobalt, chromium, cobalt/chromium alloys, magnesium, aluminum, or other biocompatible metals and or composites or alloys. Examples of other materials that may be used to form stents include carbon or carbon fiber; cellulose acetate, cellulose nitrate, silicone, polyethylene terephthalate, polyurethane, polyamide, polyester, polyorthoester, polyanhydride, polyether sulfone, polycarbonate, polypropylene, ultra-high molecular weight polyethylene, polytetrafluoroethylene, or another biocompatible polymeric material, or mixtures or copolymers of these; polylactic acid, polyglycolic acid or copolymers thereof; a polyanhydride, polycaprolactone, polyhydroxybutyrate valerate or another biodegradable polymer, or mixtures or copolymers of these; a protein, an extracellular matrix component, collagen, fibrin, or another biologic agent; or a suitable mixture of any of these.

A stent including treated ends may be fabricated to any suitable dimensions. A stent having a particular length and diameter may be selected based on the targeted vessel. For example, a stent designed for esophageal implantation may have a length ranging from about 5 cm to about 15 cm and a body diameter of about 15 mm to about 25 mm. Optionally, an esophageal stent may include one or more flanges or flares of about 10 mm to about 25 mm in length and about 20 mm to about 30 mm in diameter. A stent designed for colon implantation may have a length ranging from about 5 cm to about 15 cm and a body diameter of about 20 mm to about 25 mm. Optionally, a colonic stent may include one or more flanges having a diameter of about 25 mm to about 35 mm.

In certain embodiments a stent with treated ends may include a membrane covering over the stent framework. A stent may include covering over the entire stent framework from the proximal end to the distal end. Alternatively, the stent may have covering over a central portion of the structure but have uncovered ends or flanges. Where the stent flanges include a membrane covering, preferably the soft crowns lack membrane covering between other adjacent crowns so that the crowns may move independently from one another. Any suitable biocompatible material may be used as the membrane covering. Preferably, the membrane covering is an elastic or flexible material that can adapt to radial compression of a stent prior to delivery, as well as foreshortening of a stent during expansion from a compressed state. Suitable membrane materials include, for example, silicones (e.g. polysiloxanes and substituted polysiloxanes), polyurethanes, thermoplastic elastomers, polyolefin elastomers, polyethylene, polytetrafluoroethylene, nylon, and combinations thereof. In one preferred embodiment, the membrane covering comprises silicone. In certain embodiments, where the stent will be implanted at or near an acidic environment (e.g., will be exposed to gastric fluids), preferably the membrane covering is resistant to acid degradation.

The membrane covering may be applied to a stent by any suitable method as is known in the art. For example, the membrane may be applied by spraying, dipping, painting, brushing, or padding. Generally, the membrane covering or coating has a thickness ranging from about 0.0025 mm to about 2.5 mm, from about 0.01 mm to about 0.5 mm, or from about 0.03 mm to about 0.07 mm. The thickness of the membrane may be selected, for example, by controlling the number of dips or passes made during the application process. In one exemplary embodiment, a braided stent may be dipped in silicone liquid, removed, and thereafter cured. Preferably, the coating extends over the abluminal and luminal surfaces of the filaments, and also resides in the cells or interstices defined by the filament braid pattern.

In certain embodiments, a stent with treated ends may include one or more bioactive agents coated on the stent surfaces. A bioactive agent may be applied directly on the surface of the stent (or on a primer layer which is placed directly on the surface of the stent). Alternatively, the bioactive agent may be mixed with a carrier material and this mixture applied to the stent. In such configuration, the release of the bioactive agent may be dependent on factors including composition, structure and thickness of the carrier material. The carrier material may contain pre-existing channels, through which the bioactive agent may diffuse, or channels created by the release of bioactive agent, or another soluble substance, from the carrier material.

One or more barrier layers may be deposited over the layer containing the bioactive agent. A combination of one or more layers of bioactive agent, mixtures of carrier material/bioactive, and barrier layers may be present. The bioactive agent may be mixed with a carrier material and coated onto the stent and then over coated with barrier layer(s). Multiple layers of bioactive agent, or mixtures of carrier material/bioactive, separated by barrier layers may be present to form a multicoated stent. Different bioactive agents may be present in the different layers.

The carrier material and/or the barrier layer can include a bioelastomer, PLGA, PLA, PEG, Zein, or a hydrogel. In certain other embodiments, the carrier material and/or the barrier layer includes microcrystalline cellulose, hydroxypropylmethyl cellulose, hydroxypropyl cellulose, a cellulose product, a cellulose derivative, a polysaccharide or a polysaccharide derivative. The carrier material and/or barrier layer may include lactose, dextrose, mannitol, a derivative of lactose, dextrose, mannitol, starch or a starch derivative. The carrier material and/or barrier layer may include a biostable or a biodegradable material, for example, a biostable or biodegradable polymer.

A variety of bioactive agents may be applied to the stent in accordance with the intended use. For example, bioactive agents that may be applied include antiproliferative/antimitotic agents including natural products such as vinca alkaloids (vinblastine, vincristine, and vinorelbine), paclitaxel, rapamycin analogs, epidipodophyllotoxins (etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (for example, L-asparaginase which systemically metabolizes L-asparagine and deprives cells which do not have the capacity to synthesize their own asparagine); antiplatelet agents such as (GP) II b/IIIa inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, am inoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat); protease inhibitors; antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat.

A bioactive agent may be applied, for example, by spraying, dipping, pouring, pumping, brushing, wiping, vacuum deposition, vapor deposition, plasma deposition, electrostatic deposition, ultrasonic deposition, epitaxial growth, electrochemical deposition or any other method known to the skilled artisan.

Prior to applying a membrane, and/or a bioactive agent, a stent may be polished, cleaned, and/or primed as is known in the art. A stent may be polished, for example, with an abrasive or by electropolishing. A stent may be cleaned by inserting the stent into various solvents, degreasers and cleansers to remove any debris, residues, or unwanted materials from the stent surfaces. Optionally, a primer coating may be applied to the stent prior to application of a membrane covering, bioactive, or other coating. Preferably, the primer coating is dried to eliminate or remove any volatile components. Excess liquid may be blown off prior to drying the primer coating, which may be done at room temperature or at elevated temperatures under dry nitrogen or other suitable environments including an environment of reduced pressure. A primer layer may comprise, for example, silane, acrylate polymer/copolymer, acrylate carboxyl and/or hydroxyl copolymer, polyvinylpyrrolidone/vinylacetate copolymer (PVP/VA), olefin acrylic acid copolymer, ethylene acrylic acid copolymer, epoxy polymer, polyethylene glycol, polyethylene oxide, polyvinylpyridine copolymers, polyamide polymers/copolymers polyimide polymers/copolymers, ethylene vinylacetate copolymer and/or polyether sulfones.

A stent according to the present disclosure may be delivered to a body lumen using various techniques. Generally, under the aid of endoscopic and/or fluoroscopic visualization a delivery device containing the stent is advanced into the vicinity of the target anatomy. The targeted lumen may be predilated with a balloon catheter or other dilation device, if necessary. Preferably, the stent is delivered in a compressed state in a low profile delivery device. This approach may reduce the risk of tissue perforations during delivery. Once the delivery device is in place, the stent may be released from the retaining sheath or the like. In one preferred embodiment, a stent may be delivered with a controlled release system (e.g., Evolution™ Controlled-Release Stent, Cook Endoscopy Inc., Winston-Salem, N.C.). A controlled release device permits the physician to slowly release the stent from the retaining sheath and in some instances, recapture the stent to allow for repositioning. After implantation, the delivery device and any other devices (e.g., wire guides, catheters, etc.) may be removed.

While various embodiments of the presently disclosure have been described, it will be apparent to those of ordinary skill in the art that many more embodiments and implementations are possible within the scope of the present disclosure. Accordingly, the disclosure is not to be restricted except in light of the attached claims and their equivalents.

1. A method for treating one or more ends of a pre-woven stent to manufacture a stent, the method comprising the steps of:
providing a machine-woven stent defining a body having a tubular body portion, a first end portion, and a second end portion defined by a plurality of wires extending in a first helical direction around the body and extending in a second helical direction around the body, the first helical direction being transverse to the second helical direction, wherein the plurality of wires define a plurality of wire ends terminating at the first end portion, wherein the plurality of wires are woven together to define a plurality of cells and a plurality of intersections;
arranging the plurality of wires in a plurality of wire pairs comprising a first wire and second wire at the first end portion of the machine-woven stent;
applying a cap to the wire ends of each the plurality of wire pairs, wherein the cap has a first end for being attached to the first wire and a second end for being attached to the second wire;
fixing the first end of the cap to the first wire; and
fixing the second end of the cap to the second wire.

2. The method of claim 1, further comprising trimming the wire pairs prior to applying the cap.

3. The method of claim 1, further comprising unweaving the plurality of wires prior to arranging the plurality of wires in wire pairs.

4. The method of claim 1, wherein the caps attached to each of the plurality of wire pairs are disposed at the same axial location.

5. The method of claim 1, wherein a first set of the caps attached to the wire pairs are disposed at a first axial location and a second set of the caps attached to the wire pairs are disposed at a second axial location that is different from the first axial location.

6. The method of claim 1, wherein the cap is in the form of a bent cannula.

7. The method of claim 1, wherein the cap is in the form of a pair of cannulas connected by a flexible bridge portion.

8. The method of claim 1, wherein the cap is in the form of a microcoil.

9. The method of claim 7, wherein the flexible bridge portion is in the form of a wire.

10. The method of claim 9, wherein the wire is coiled into a microcoil.

11. The method of claim 1, wherein the cap is in the form of a flexible heat shrink tube, and the ends of the first and second wires are secured to cap by heating the tube.

12. The method of claim 1, wherein the second end portion of the machine woven stent includes multiple crowns without caps.

13. The method of claim 1, further comprising applying the caps to a plurality of wire pairs at the second end portion of the machine woven stent in addition to the first end portion.

14. The method of claim 1, further comprising:
providing a mandrel on which the machine woven stent is woven, wherein the mandrel includes a body portion and a head portion, the head portion disposed at the first end portion of the stent;
separating the body portion from the head portion after applying the caps at the first end portion;
removing the body portion from the stent and leaving the head portion in place:
inserting a reduced diameter portion mandrel having a reduced diameter relative to the head portion into the stent;
heat setting the stent on the reduced diameter portion mandrel and the head portion; and
defining a flanged portion and a body portion of the stent.

15. The method of claim 14, wherein the mandrel includes a second head portion at the second end portion of the stent, further comprising collapsing the body portion and removing the body portion from the stent and leaving the first and second head portions in place.

16. A pre-woven stent having treated ends, the stent comprising:
a body having a tubular body portion, a first end portion, and a second end portion defined by a plurality of wires extending in a first helical direction around the body and extending in a second helical direction around the body, the first helical direction being transverse to the second helical direction, wherein the plurality of wires define a plurality of wire ends terminating at the first end portion, wherein the plurality of wires are woven together to define a plurality of cells and a plurality of intersections;
wherein the plurality of wire ends are arranged in wire pairs comprising a first wire and a second wire, wherein for each wire pair, the first wire is connected to the second wire;
a plurality of caps, each cap of the plurality of caps connecting the first wire to the second wire for each wire pair;
where the first wire and second wire extend in the same axial direction away from the stent body;
wherein the cap has a first end for being attached to the first wire and a second end for being attached to the second wire, and the cap extends from the first end in the same axial direction as the first wire, curves around a bend point, and extends from the bend point toward the second end in the opposite axial direction as the second wire;
wherein the first wire is received in the first end of the cap and fixed in place relative to the cap, and the second wire is received in the second end of the cap and fixed in place relative to the cap.

17. The stent of claim 16, wherein the first wire and the second wire do not overlap.

18. The stent of claim 16, wherein the cap is in the form of a thin walled cannula.

19. The stent of claim 16, wherein the cap is in the form of a pair of cannulas connected by a flexible bridge portion.

20. The stent of claim 16, wherein the plurality of caps are disposed at different axial locations.
